(19) United States
(12) Patent Application Publication
(10) Pub. No.: US 2024/0144552 A1
(43) Pub. Date: May 2, 2024

(54) IMAGE RECONSTRUCTION DEVICE, IMAGE RECONSTRUCTION METHOD AND IMAGE RECONSTRUCTION SYSTEM FOR MONITORING A MEDICAL OR A COSMETIC SKIN CONDITION

(71) Applicants: DERMATOO, Chaudfontaine (BE); IOT-D, Liege (BE)

(72) Inventors: Vincent Joassin, Comblain-au-pont (BE); Jeremie Bours, Comblain-au-pont (BE); Romain Bajoit, Annut (BE); Julien Delarbre, Embourg (BE); Maxime Noirhomme, Limerle (BE); Sebastien Blondiau, Liege (BE); Robin Murgira, La Roche-en-Ardenne (BE); Francois Delarbre, Wasseiges (BE); Francois Royen, Donceel (BE); Maxime Aerts, Retinne (BE)

(21) Appl. No.: 18/278,873

(22) PCT Filed: Jan. 24, 2022
(86) PCT No.: PCT/EP2022/051517

§ 371 Date: Aug. 25, 2023

(30) Foreign Application Priority Data
Feb. 25, 2021 (BE) BE2021/5132

(51) Int. Cl. G06T 11/00; G06T 5/50; G06T 15/00; G06V 10/143; G06V 10/25

(52) U.S. Cl. CPC G06T 11/005; G06T 5/50; G06T 15/00; G06V 10/143; G06V 10/25; G06T 2207/20221

(57) ABSTRACT

An image reconstruction device, image reconstruction method and image reconstruction system for monitoring a medical or cosmetic skin condition wherein the image reconstruction device includes a camera, a calibration tube, a wireless communication system and a graphical user interface. The calibration tube (allows for the controlled reconstruction of a calibration image) of the surrounding tissue of a region of interest of the skin at a calibration distance. A panoramic image of the region of interest of the skin is registered without the calibration tube at a panoramic distance. A near-distance image of the region of interest is registered at a near distance. Additional information about the region of interest of the skin may be registered through one or more sensors. The calibration image, the panoramic image and the near-distance image are calibrated and superposed to obtain a three-dimensional image useful in the monitoring of a medical or cosmetic skin condition.

### BACKGROUND

This invention relates to an image reconstruction device (100), image reconstruction method and image reconstruction system for monitoring a medical or cosmetic skin condition.

Images of a medical or cosmetic skin condition may vary significantly. Major variation factors include the brightness, the distance and the image angle. Therefore, the present invention aims at providing calibrated image reconstructions of medical or cosmetic skin conditions in spite of these disturbing variations.

### PRIOR ART

EP3559741 to Esthetic Visual Analytics discloses a camera comprising two imaging systems each comprising a different optical path corresponding to a different viewing angle of an object. The camera further comprises two masks comprising multiple apertures, a detector and a processor configured to produce a multi-layer three dimensional reconstruction of the object. However, no calibration is described.

### SHORT DESCRIPTION OF THE INVENTION

The present inventors surprisingly found that varying light conditions, distances between the camera focus point and the region of interest of the skin, and angles in biomedical images of the skin can be calibrated by using a calibration tube (10) on the camera (20) of an image reconstruction device (100). The image reconstruction device (100) is configured to obtain a calibration image (50) with defined imaging conditions at a calibration distance. The calibration image (50) is an image of the calibration region (55). The calibration region (55) neighbors the region of interest (200). However, the calibration image (50) does not comprise the region of interest (200). The image reconstruction device (100) is further configured to obtain a panoramic image (60) of the panoramic region (65). The panoramic region (65) is obtained at a distance large enough to register both the calibration region (55) and the region of interest (200). A near-distance image (70) is obtained at a near distance. The near-distance image (70) does not comprise or only partially comprise the calibration region (55) corresponding to the calibration image (50). The near distance image (70) contains information corresponding to the near-distance region (75). The image data may be used to reconstruct a calibrated superposed multi-layer image of the region of interest (200).

Accordingly, a first aspect of the invention is an image reconstruction device (100) comprising:
A camera (20) comprising one or more objective lenses;
A detector configured to acquire a plurality of images of a region of interest (200) of the skin;
A processor configured to produce from the image information, which is obtained by the camera (20), a multilayer three dimensional reconstruction of the region of interest (200) of the skin;
A graphical user interface (30) to display the image information;
A wireless communication system to transmit the image information obtained by the detector;
An energy source;
wherein a calibration tube (10) is mounted on the camera (20) of the image reconstruction device (100);
wherein the image reconstruction device (100) is configured to obtain
a calibration image (50) of the calibration region (55) in proximity of the region of interest (55) through the calibration tube (10) at a calibration distance;
a panoramic image (60) of the panoramic region (65) at a panoramic distance;
a near-distance image (70) of the region of interest (200) at a near distance; and
wherein the calibration region (65) comprises the region of interest (200) and the calibration region (55);
wherein the near-distance image (70) fully or partially excludes the calibration region (55);
wherein the image reconstruction device (100) is further configured to fuse the calibration image (50), the panoramic image (60) and the near-distance image (70) to obtain a calibrated superposed multi-layer reconstruction of the region of interest of the skin (200).

In another embodiment, the calibration tube (10) is opaque or partially or fully transparent.

In another embodiment, the calibration image is an image of the tissue surrounding the region of interest (200).

In another embodiment, the calibration image (50) is an image of the tissue surrounding the region of interest (200).

In another embodiment, the panoramic image (60) comprises the skin tissue corresponding to the calibrating image (50) and the region of interest (200).

In another embodiment, the calibration tube (10) has a length of
1 cm or more, preferably of 2.5 cm or more, even more preferably of 5 cm or more, or of
15 cm or less, preferably of 10 cm or less, even more preferably of 7.5 cm or less.

In another embodiment, the image reconstruction device (100) further comprises a light source.

In another embodiment, the light source is an adaptable LED.

In another embodiment, the image reconstruction device (100) further comprises a voice recording system (40).

In another embodiment, the image reconstruction device (100) further comprises a infrared or violet light emission source.

In another embodiment, the image reconstruction device (100) further comprises one or more filters comprising a plurality of apertures.

In another embodiment, the image reconstruction device (100) further comprises a sensor for measuring the temperature or the presence or quantity of bacteria in the region of interest (200).

A further aspect of the invention is the use of an image reconstruction device (100) for monitoring a medical or cosmetic skin condition over time.

Another aspect of the invention is the use of the image reconstruction device as a decision aid for medical or cosmetic professionals.

A further object of the invention is an image reconstruction method comprising the following steps:
Providing an image reconstruction device (100) of the invention;
Obtaining a calibration image (50) of the calibration region (55) in proximity of the region of interest (55) through the calibration tube (10) at a calibration distance;
Obtaining a panoramic image (60) of the panoramic region (65) at a panoramic distance;
Obtaining a near-distance image (70) of the region of interest (200) at a near distance;
Fusing the calibration image (50), the panoramic image (60) and the near-distance image (70) to obtain a calibrated superposed multi-layer reconstruction of the region of interest (200);
wherein the panoramic region (75) comprises the region of interest (200) and the calibration region (55); and
wherein the near-distance image (70) is configured to fully or partially exclude the calibration region (55).

A further aspect of the invention is an image reconstruction system comprising the image reconstruction device (100) of the invention and configured to perform the image reconstruction method of the invention.

### SHORT DESCRIPTION OF THE DRAWINGS

FIG. 1 shows a schematic illustration of the image reconstruction device (100) of the invention comprising a calibration tube (10) to a obtain a calibration image (50).

FIG. 2 shows a schematic illustration of the image reconstruction device (100) and a panoramic image (60) of the region of interest (200) that comprises the region of the skin corresponding to the calibration image (50).

FIG. 3 shows a schematic illustration of the image reconstruction device (100) and a near-distance image (70) of the region of interest (200) but not, or only partially the calibration region (55).

### DETAILED DESCRIPTION OF THE DRAWINGS

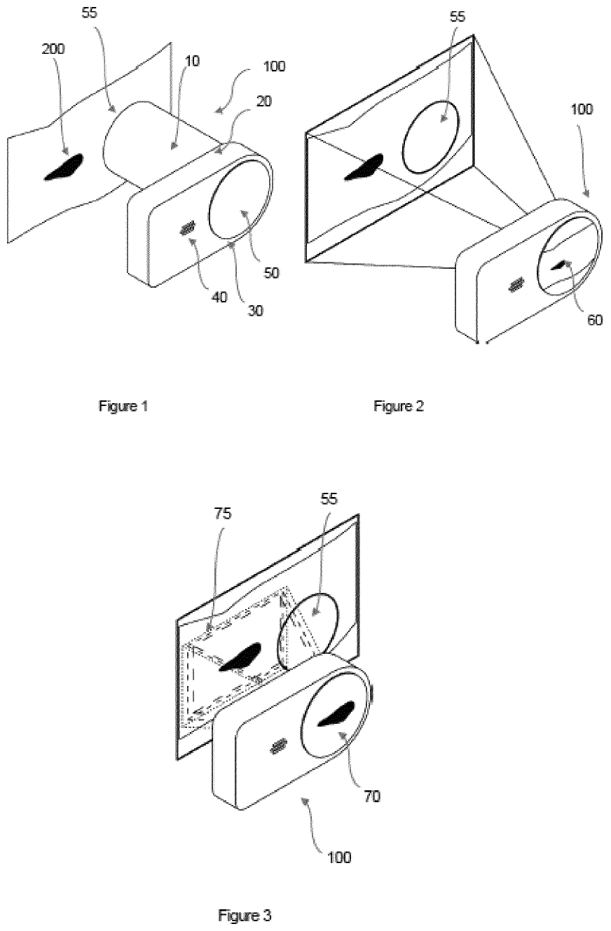

FIG. 1, FIG. 2 and FIG. 3 show an exemplary image reconstruction device (100) of the invention comprising:
A camera (20) comprising one or more objective lenses;
A detector configured to acquire a plurality of images of a region of interest (200) of the skin;
A processor configured to produce from the image information obtained by the one or more imaging systems a multilayer three dimensional reconstruction of the region of interest (200) of the skin;
A graphical user interface (30) to display the image information;
A wireless communication system to transmit the image information obtained by the detector; and
An energy source.

A calibration tube (10) is mounted on the camera (20) of the image reconstruction device (100). The image reconstruction device (100) is configured to obtain a calibration image (50) of the calibration region (55) in proximity of the region of interest (55) through the calibration tube (10) at a calibration distance as shown in FIG. 1. The calibration region usually covers parts of the surrounding skin tissue of the region of interest (200). However, it does not comprise the region of interest (200).

The image reconstruction device (100) is further configured to obtain a panoramic image (60) of the panoramic region (65) at a panoramic distance as shown in FIG. 2.

The image reconstruction device (100) is further configured to obtain a near-distance image (70) of the region of interest (200) at a near distance as shown in FIG. 3. The near distance image (70) fully or partially excludes the calibration region (55).

The image reconstruction device (100) is further configured to fuse the calibration image (50) of FIG. 1, the panoramic image (60) of FIG. 2 and the near-distance image (70) of FIG. 3 to obtain a calibrated superposed multi-layer reconstruction of the region of interest (200).

The image reconstruction device is useful in monitoring a region of interest (200) of the skin, preferably a lesion, a pathological tissue or wound, or the surrounding tissue.

Camera (20)

The camera (20) may be any suitable camera. In one embodiment, the camera is capable of registering a three-dimensional image of the region of interest (200).

Calibration Tube (10) for Controlled Environment

The calibration tube (10) enables a controlled environment for the registration of the calibration image (50). Controlled environment is understood as a region under the calibration tube (10) in which the brightness can be adjusted, hereinafter referred to as calibration region (55). The calibration tube (10) may be opaque or fully or partially transparent. The image registered under calibration tube (10) serves as a reference for the calibration of the near-distance image (70). Calibration depends on the type of test pattern used or on the digital reconstruction. The calibration image (10) is obtained at a calibration distance, a calibration angle and a calibration lighting condition.

The calibration tube (10) is fixed to the camera (20) of the image reconstruction device (100) in a way to from a tunnel from the optics in the camera towards the calibration area (55) of the skin. The fixation of the calibration tube (10) to the camera of the image reconstruction device (100) may be permanent, rotatable or removable. In one embodiment, the calibration tube (10) is removable, cleanable and reusable.

The length of the calibration tube corresponds to the calibration distance. In one embodiment, the calibration distance is 1 cm or more, preferably of 2.5 cm or more, even more preferably of 5 cm or more. In another embodiment, the calibration distance is 15 cm or less, preferably of 10 cm or less, even more preferably of 7.5 cm or less.

In one embodiment, the calibration tube (10) is extendable.

In another embodiment, the calibration tube (10) is removably or rotatably attached to the image reconstruction device (100).

The form of the calibration tube (10) may vary. In one embodiment, the calibration tube (10) is round or in the form of a hollow cylinder. However, the calibration tube (10) may be any other suitable hollow body. For example the calibration tube (10) may have a polygonal, quadrangular or rectangular cross section. The calibration tube (10) may have a symmetrical cross section or an asymmetrical cross section. The cross section may taper off or may widen.

Calibration

The image reconstruction device (100) is further configured to fuse the calibration image (50), the panoramic image (60) and the near-distance image (70) to obtain a calibrated superposed multi-layer reconstruction of the region of interest (200). This fusion step may be performed in the image reconstruction device (100) or remotely for example in a cloud system, for example in a protected medical cloud system.

In one embodiment, the calibration is performed through machine learning, wherein one or more of the calibration image (50), the panoramic image (60), and the near-distance image (70) are deformed.

In another embodiment, convolutional neural networks are used for the deformation of the calibration image (50), the panoramic image (60), and the near-distance image (70). The network is trained on the calibration image (50), the panoramic image (60), and the near-distance image (70) of a region of interest (200). Typically, for training purposes, the region of interest (200) is a wound or a lesion that is monitored over time.

Sensors

The image reconstruction device (100) may have one or more sensors for measuring other parameters of the region of interest (200) for example temperature or for measuring the presence, type or quantity of bacteria.

Violet Light Emission and Filters

In another embodiment, the image reconstruction device (100) further comprises a violet light emission source.

In another embodiment, the violet light is 100 nm to 600 nm, preferably 200 nm to 500 nm and even more preferably, 350 nm to 450 nm. Violet light is used to detect bacteria and other features of interest, for example moist, blood, dirt or external bodies in the region of interest (200).

In another embodiment, a filter is mounted on the camera (20).

Graphical User Interface (30)

The graphical user interface (30) may be any system capable of displaying image information.

Voice Recording System (40)

The image reconstruction device (100) may have a voice recording system (40). The voice recording system (40) enables the reconstruction of voice messages that may be transmitted through the wireless communication system. Consequently, medical professionals may provide remote consultations on a medical or cosmetic skin condition based on the image and the voice information obtained and transmitted by the image reconstruction device (100).

Wireless Communication System

The wireless communication system may be any wireless communication system capable of transmitting the image information. The wireless communication system allows for example for medical professionals to remotely obtain a calibrated image of a region of interest (200), in particular a medical or cosmetic skin condition.

Three-Dimensional Imaging and Reconstruction of Skin Lesion

The image reconstruction device (100) is particular useful in three-dimensional imaging and reconstruction of images of skin lesions or wounds.

Image Reconstruction Method

The image reconstruction method of the invention comprising the following steps:
Providing an image reconstruction device (100) of the invention;
Obtaining a calibration image (50) of the calibration region (55) in proximity of the region of interest (55) through the calibration tube (10) at a calibration distance;
Obtaining a panoramic image (60) of the panoramic region (65) at a panoramic distance;
Obtaining a near-distance image (70) of the region of interest (200) at a near distance;
Fusing the calibration image (50), the panoramic image (60) and the near-distance image (70) of the region of interest (200) to obtain a calibrated superposed multi-layer reconstruction of the region of interest (200).
wherein the panoramic region (75) comprises the region of interest (200) and the calibration region (55); and
wherein the near-distance image (70) is configured to fully or partially exclude the calibration region (55).

In another embodiment, the image reconstruction method further comprises the step of obtaining the temperature of the region of interest (200) through a temperature sensor in the image reconstruction device (100).

In another embodiment, the image reconstruction method further comprises the step of obtaining a filtered image through a violet light emission source and filters mounted on the camera (20) of the image reconstruction device (100).

The image reconstruction method enables a medical professional to obtain image, the temperature of the region of interest (200) and voice information in respect of a medical or cosmetic skin condition remotely and in real time and thus to choose the appropriate treatment.

Monitoring a Medical or Cosmetic Skin Condition

The image reconstruction device (100), image reconstruction method and image reconstruction system may be used to monitor a region of interest (200), in particular a medical or cosmetic skin condition. Monitoring a medical or cosmetic skin condition means providing information at different points in time about the healing process, the evolution or development of a medical or cosmetic skin condition or the effectiveness of a treatment of a medical or cosmetic skin condition. Exemplary medical or cosmetic skin conditions include lesions, wounds or any other pathological conditions of the skin. It may also be used to support treatment decisions.

1. Image reconstruction device (100) comprising
A camera (20) comprising one or more objective lenses;
A detector configured to acquire a plurality of images of a region of interest (200) of the skin;
A processor configured to produce from the image information obtained by the one or more imaging systems a multilayer three dimensional reconstruction of the region of interest (200) of the skin;
A graphical user interface (30) to display the image information;
A wireless communication system to transmit the image information obtained by the detector;
An energy source;
wherein a calibration tube (10) is mounted on the camera (20) of the image reconstruction device (100);
wherein the image reconstruction device (100) is configured to obtain
a calibration image (50) of the calibration region (55) in proximity of the region of interest (200) through the calibration tube (10) at a calibration distance;
a panoramic image (60) of the panoramic region (65) at a panoramic distance;
a near-distance image (70) of the region of interest (200) at a near distance;
wherein the panoramic region (65) comprises the region of interest (200) and the calibration region (55);
wherein the near-distance image (70) fully or partially excludes the calibration region (55);
wherein the image reconstruction device (100) is further configured to fuse the calibration image (50), the panoramic image (60) and the near-distance image (70) to obtain a calibrated superposed multi-layer reconstruction of the region of interest (200).

2. Image reconstruction device (100) according to claim 1, wherein the calibration tube (10) is an opaque or partially or fully transparent hollow body.

3. Image reconstruction device (100) according to claim 1, wherein the calibration image (50) is an image of the tissue surrounding the region of interest (200).

4. Image reconstruction device (100) according to claim 1, wherein the panoramic image (60) comprises the skin tissue corresponding to the calibrating image (50) and the region of interest (200).

5. Image reconstruction device (100) according to claim 1, wherein the calibration tube (10) has a length of
1 cm or more, preferably of 2.5 cm or more, even more preferably of 5 cm or more, or of
15 cm or less, preferably of 10 cm or less, even more preferably of 7.5 cm or less.

6. Image reconstruction device (100) according to claim 1, wherein the image reconstruction device (100) further comprises a light source, in particular an adaptable LED.

7. Image reconstruction device (100) according to claim 1, wherein the image reconstruction device (100) further comprises a voice recording system (40).

8. Image reconstruction device (100) according to claim 1, wherein the one or more light sources are infrared or violet light emission sources.

9. Image reconstruction device (100) according to claim 1, wherein the image reconstruction device (100) further comprises one or more filters comprising a plurality of apertures.

10. Image reconstruction device (100) according to claim 1, wherein the image reconstruction device (100) further comprises a sensor for measuring the temperature or the presence and quantity of bacteria in the region of interest (200).

11. Process for monitoring a medical or cosmetic skin condition, wherein the monitoring of the medical or cosmetic skin condition is performed with an image reconstruction device (100) according to claim 1.

12. Image reconstruction method comprising the following steps:
Providing an image reconstruction device (100) according to any of the preceding claims;
Obtaining a calibration image (50) of the calibration region (55) in proximity of the region of interest (55) through the calibration tube (10) at a calibration distance;
Obtaining a panoramic image (60) of the panoramic region (65) at a panoramic distance;
Obtaining a near-distance image (70) of the region of interest (200) at a near distance;
fusing the calibration image (50), the panoramic image (60) and the near-distance image (70) of the region of interest (200) to obtain a calibrated superposed multi-layer reconstruction of the region of interest (200).
wherein the panoramic region (75) is configured to comprise both the region of interest (200) and the calibration region (55); and
wherein the near-distance image (70) is configured to fully or partially exclude the calibration region (55).

13. Image reconstruction system comprising the image reconstruction device (100) according to claim 1 and configured to perform the image reconstruction method of claim 12.
